(19) United States
(12) Patent Application Publication
(10) Pub. No.: US 2005/0142174 A1
(43) Pub. Date: Jun. 30, 2005

(54) Adhesive and binding agent for dermal or transdermal treatment systems

(75) Inventors: Manfred Assmus, Bickenbach (DE); Erna Roth, Darmstadt (DE); Antoni Boix, St. Joan (ES); Isabel Reig, St. Joan Despi (ES)

Correspondence Address: OBLON, SPIVAK, MCCLELLAND, MAIER & NEUSTADT, P.C., 1940 DUKE STREET, ALEXANDRIA, VA, 22314, US

(73) Assignee: Roehm GMBH & Co. KG

(21) Appl. No.: 10/499,818

(22) PCT Filed: Dec. 19, 2002
(86) PCT No.: PCT/EP02/14556

(30) Foreign Application Priority Data
Jan. 9, 2002 (DE) 10200578.8

(51) Int. Cl. A61K 31/485; A61K 9/70

(52) U.S. Cl. 424/449; 514/282

(57) ABSTRACT

The invention relates to an adhesive and binding agent for dermal or transdermal treatment systems, said agent containing (a) a (meth)acrylate copolymer consisting of radically polymerised C<sb>1</sb>to C<sb>4</sb>alkyl esters of acrylic or methacrylic acid and (meth)acrylate monomers having a cationic ammonium group in the alkyl radical; (b) between 0.1 and 45 wt. %, in relation to (a), of an organic dicarboxylic or tricarboxylic acid, or of acrylate or (meth)acrylate polymers or copolymers containing acid groups; (c) a skin penetration enhancer; and (d) optionally a pharmaceutical active ingredient, a softener and/or at least one standard addition agent. The inventive adhesive and binding agent is characterised in that the penetration enhancer (c) is an alcohol comprising between 10 and 12 carbon atoms.

The invention relates to an adhesive and binder for dermal or transdermal therapeutic systems. Prior art

EP-A 315 218 describes pharmaceutical compositions intended for the systemic transdermal administration of active pharmacological substances and characterized in that the active pharmacological substances are located in a reservoir that comprises a polyacrylate polymer having cationic properties. Plasticizers and can be present in amounts of from 3 to 33% by weight. Penetration enhancers can be present in amounts from 10 to 100% by weight. The pharmaceutical composition may further be provided with an adhesive layer in order to achieve effective adhesion to the skin.

EP-A 617 972 describes laminar dermal therapeutic systems with retarded release of active substance which are composed of mixtures of poly(meth)acrylates and are prepared from a melt. One poly(meth)acrylate component contains functional groups while another poly(meth)acrylate component contains insignificant amounts of functional groups, if any, and essentially regulates the flow behavior of the polymeric adhesive layer. Penetration enhancers are mentioned as possible additives.

EP-A 848 950 and EP-A 848 960 describe adhesives and binders for pharmaceutical purposes that are distinguished by high hydrophilicity and/or high water vapor permeability and at the same time combine a high bond strength with low cold flow. The adhesives and binders are therefore outstandingly suitable as pressure-sensitive skin adhesives or transdermal therapeutic systems.

EP-A 0 848 960 describes an adhesive and binder for dermal or transdermal therapeutic systems which consists of (a1) 55-99.9% by weight of a (meth)acrylate copolymer formed from structural and functional monomers, the functional monomers containing tertiary or quaternary amino groups, (a2) 0.1-45% by weight of an acid-functional acrylate or (meth)acrylate polymer or copolymer, and (b) 25-80% by weight, based on the sum of (a1) and (a2), of a plasticizer.

EP-A 848 950 describes an adhesive and binder for dermal or transdermal therapeutic systems which consists of (a) 85-99% by weight of a (meth)acrylate copolymer formed from structural and functional monomers, the functional monomers containing tertiary or quaternary amino groups, (b) 15-0.1% by weight of an organic dicarboxylic or tricarboxylic acid and (c) 40-70% by weight, based on the sum of (a) and (b), of a plasticizer.

DE 40 20 144 C2 describes a device for the topical and systemic administration of active substances. The systems in question are mixtures of polyacrylate adhesive with a film former, e.g., a neutral copolymer of methyl methacrylate and butyl methacrylate (PLASMID® B), which may comprise plasticizing auxiliaries such as solubilizers or penetration enhancers, e.g., fatty alcohols such dodecanol, in amounts of, for example, 20% by weight.

Problem and Solution

A fundamental issue affecting adhesives and binders for dermal or transdermal therapeutic systems is the harmonization of the components in such a way that a number of parameters are met simultaneously. Suitable methods of determining these parameters, which are specified below, are familiar to the skilled worker in the field of dermal or transdermal therapeutic systems (on this point see also the remarks in the “Examples” section).

Mention should be made first of all of a sufficient bond strength, which ought though to be only so high that when the adhesive layer is detached there is adhesive fracture but not cohesive fracture.

In addition there are minimum requirements to be met in terms of the properties of tack and cold flow. The tack ought generally to be at least 1 and the cold flow not more than 1 mm/24 hours.

Finally, an active pharmaceutical substance present ought to penetrate the skin effectively. Penetration figures of at least 1 μg active substance/cm² are the target.

The latter objective in particular is difficult to achieve, since it requires the addition of comparatively high proportions of penetration enhancers, which are known per se. The relatively high proportions required have an effect on the overall system, alongside other requisite components such as plasticizers, which is generally negative. If, on the other hand, the amount of other components, such as the amount of the plasticizer, for example, is lowered in favor of the penetration enhancer, the desired penetration rate is achieved but the other properties, particularly the bond strength, are then generally no longer within the target range.

The problem was therefore seen to be to provide an adhesive and binder for dermal or transdermal therapeutic systems that as far as possible meets all of the abovementioned criteria simultaneously. A further target ought to be to lower the amount of plasticizing substances which do not enhance penetration, as far as is possible, or to do without them entirely, so that the amount of the other ingredients and additives does not become too high with respect to the amount of the actual matrix-forming system, so that, for example, controlled release of active substance is unaffected.

Surprisingly it has been found that the problem is solved by means of

an adhesive and binder for transdermal therapeutic systems, comprising

(a) a (meth)acrylate copolymer of free-radically polymerized C₁ to C₄ alkyl esters of acrylic or methacrylic acid and (meth)acrylate monomers having a cationic ammonium group in the alkyl radical, comprising

(b) 0.1-45% by weight based on (a) of an organic dicarboxylic or tricarboxylic acid or of an acid- functional acrylate or (meth)acrylate polymer or copolymer, and

(c) a skin penetration enhancer

(d) if desired, an active pharmaceutical substance, a plasticizer and/or one or more customary pharmaceutical additives

characterized in that

the penetration enhancer (c) is an alcohol having 10 to 12 carbon atoms.

### Implementation of the Invention

Component (a)

Component (a) is a (meth)acrylate copolymer composed of free-radically polymerized C₁ to C₄ alkyl esters of acrylic or methacrylic acid and (meth)acrylate monomers having a cationic ammonium group in the alkyl radical. This definition embraces copolymers which, under product names including EUDRAGIT® E, EUDRAGIT® RS or EUDRAGIT® RL, have been known for a long time as drug coatings.

The amount of the (meth)acrylate monomers having a cationic ammonium group in the alkyl radical is preferably at least 2%, more preferably at least 4% by weight based on the copolymer.

C₁ to C₄ alkyl esters of acrylic or methacrylic acid are, in particular, methyl acrylate, ethyl acrylate, butyl acrylate, butyl methacrylate, and methyl methacrylate.

(Meth)acrylate monomers having a cationic ammonium group in the alkyl radical are, in particular, (meth)acrylate monomers having tertiary or quaternary amino and/or ammonium groups in the alkyl radical.

The amount of the functional monomers having tertiary ammonium groups can advantageously be from 30 to 70% by weight, preferably from 40 to 60% by weight. Suitable monomers having tertiary ammonium groups are listed in US 4 705 695, column 3, line 64 to column 4, line 13. Mention may be made in particular of dimethylaminoethyl acrylate, 2-dimethylaminopropyl acrylate, dimethylaminopropyl methacrylate, dimethyl-aminobenzyl acrylate, dimethylaminobenzyl methacrylate, (3-dimethylamino-2,2-dimethyl)propyl acrylate, dimethyl-amino-2, 2-dimethyl)propyl methacrylate, (3-diethylamino-2, 2-dimethyl)propyl acrylate and diethylamino-2,2-dimethyl)propyl methacrylate. Particular preference is given to dimethylaminoethyl methacrylate.

A (meth)acrylate copolymer corresponding to component (a) and containing tertiary amino groups may be synthesized, for example, from 20-30% by weight of methyl methacrylate, 20-30% by weight of butyl methacrylate, and 60-40% by weight of dimethylaminoethyl methacrylate.

A (meth)acrylate copolymer corresponding to component (a) and containing tertiary amino groups may be synthesized, for example, from 25% by weight of methyl methacrylate, 25% by weight of butyl methacrylate, and 50% by weight of dimethylaminoethyl methacrylate (EUDRAGIT® E 100).

The amount of the functional monomers having quaternary ammonium groups can be preferably from 2 to 15%, more preferably from 4 to 12% by weight. As a monomer having functional quaternary ammonium groups particular preference is given to 2-trimethylammonioethyl methacrylate chloride.

Corresponding (meth)acrylate copolymers are known for example from EP-A 181 515 or DE-C 1 617 751. These are polymers which are swellable or soluble independently of the pH and which are suitable for drug coatings. A possible preparation process is that of bulk polymerization in the presence of a radical-forming initiator in solution in the monomer mixture. The polymer may also be prepared by means of solution or precipitation polymerization. In this way the polymer may be obtained in the form of a fine powder, which in the case of bulk polymerization can be achieved by grinding and in the case of solution and precipitation polymerization by spray drying, for example. A corresponding copolymer may be synthesized, for example, from 50-70% by weight of methyl methacrylate, 20-40% by weight of ethyl acrylate, and 7-2% by weight of 2-trimethylammonioethyl methacrylate chloride.

Another suitable (meth)acrylate copolymer may be synthesized, for example, from 85 to less than 93% by weight of C1 to C4 alkyl esters of acrylic or methacrylic acid and more than 7 to 15% by weight of (meth)acrylate monomers having a quaternary ammonium group in the alkyl radical. (Meth)acrylate monomers of this kind are commercially customary and have long been used for retardant coatings.

A (meth)acrylate copolymer corresponding to component (a) and containing quaternary amino groups may be synthesized, for example, from 60% by weight of methyl methacrylate, 30% by weight of ethyl acrylate, and 10% by weight of 2-trimethylammonioethyl methacrylate chloride (EUDRAGIT® RL 100).

A further preferred (meth)acrylate copolymer corresponding to component (a) and containing quaternary amino groups may be synthesized, for example, from 65% by weight of methyl methacrylate, 30% by weight of ethyl acrylate, and 5% by weight of 2-trimethylammonioethyl methacrylate chloride (EUDRAGIT® RS 100).

The copolymers (a) are obtained in conventional manner by free-radical bulk, solution, bead or emulsion polymerization. They may be in the form of extruded granules, ground powder, solution or dispersion.

Component (b)

Component (b) functions as a counterion to the cationic component (a). Component (b) can be adjusted so that partial or complete neutralization is effected. The cationic radicals of the cationic (meth)acrylate copolymer (a) are neutralized to an extent of preferably from 2 to 100%, more preferably from 5 to 60 by means of component (b).

Component (b) is composed, based on component (a), of from 0.1 to 45%, preferably from 1 to 30%, more preferably from 5 to 25% by weight, based on (a), of an organic dicarboxylic or tricarboxylic acid or of an acid-functional acrylate or (meth)acrylate polymer or copolymer. Preference is given to using an organic dicarboxylic or tricarboxylic acid, since its proportion be restricted generally to 0.1 to 18%, preferably 5 to 15% by weight.

Suitability is possessed by organic dicarboxylic tricarboxylic acids, preferably succinic acid (succinate), fumaric acid or citric acid.

A further suitable component (b) are acid-functional acrylate or (meth)acrylate polymers or copolymers.

Suitability is possessed, for example, by polyacrylic acid (®Carbopol).

Preference, however, is given to copolymers of structural and functional (meth)acrylate monomers. Structural acrylic or methacrylate monomers are, for example, C₁ to C₄ alkyl esters of acrylic or methacrylic acid. Preference is given to methyl acrylate, ethyl acrylate, butyl acrylate, and methyl methacrylate. As a monomer with functional acid groups methacrylic acid is particularly preferred.

A copolymer corresponding to component (b) may be synthesized, for example, from 30-70% by weight of ethyl acylate or methyl methacrylate and 70-30% by weight of methacrylic acid.

It is essential for the present invention that components (a) and (b) are present in the stated proportions. If the fraction of the acid-functional copolymer (b) is less than 0.1% by weight then the cold flow is generally too high. The disadvantage of a fraction of more than 45% by weight is that the processing properties are affected.

Component (c)

Component (c) is a penetration promoter which is an alcohol having 10 to 12 carbon atoms. Component (c) can for example is decanol, or n-decanol, undecanol (1-undecanol or 2-undecanol) or dodecanol, or n-dodecanol.

Active Pharmaceutical Substances

Important examples of suitable active pharmaceutical substances (groups and individual substances), without any claim to completeness, are the following:
analgesics
antiallergics, antiarrhythmics,
antibiotics, chemotherapeutics, antidiabetics, antidotes,
antiepileptics, antihypertensives, antihypotensives,
anticoagulents, antimycotics, anti-inflammatories,
beta-receptor blockers, calcium antagonists and ACE inhibitors,
broncholytics/antasthmatics, cholinergics, corticoids (internals),
dermatics, diuretics, enzyme inhibitors, enzyme preparations and transport proteins,
expectorants, geriatric agents, gout agents, influenza agents,
hormones and inhibitors thereof, hypnotics/sedatives, cardiac agents, lipid reducers,
parathyroid hormones/calcium metabolism regulators,
psychopharmaceuticals, sex hormones and their inhibitors,
spasmolytics, sympatholytics, sympathomimetics, vitamins,
wound treatment agents, and cytostatics.
   Examples of Active Substances are:

The invention is particularly suitable for the provision of drug forms comprising the active substances below.

Therapeutic Categories:

analgesics, antirheumatics, antiallergics, antiarrhythmics, beta-receptor blockers, calcium channel blockers, inhibitors of the renin-angiotensin system, broncholytics/antasthmatics, cholinergics, diuretics, circulation promoters, gout agents, influenza agents, coronary agents, lipid reducers, gastrointestinal agents, psychopharma-ceuticals, platelet aggregation inhibitors, urological agents, venous therapeutic agents, vitamins, and minerals

Active Substances

Morphine and/or its derivatives, tramadol, acetylsalicylic acid, diclofenac, indometacin, lonazolac, ibuprofen, ketoprofen, propyphenazone, naproxen, paracetamol, flurbiprofen, dimetindene, quinidine, metoprolol, propranolol, oxprenolol, pindolol, atenolol, metoprolol, disopyramide, verapamil, diltiazem, gallopamil, nifedipine, nicardipine, nisoldipine, nimodipine, amlodipine, theophylline, salbutamol, sildenafil, terbutaline, ambroxol, aminophylline, choline theophyllinate, pyridostigmine, piretanide, furosemide, pentoxifylline, naftidrofuryl, buflomedil, xantinol nicotinate, bencyclane, allopurinol, norephedrine, clorphenamine isosorbide mononitrate, isosorbide dinitrate, glyceryl trinitrate, molsidomine, bezafibrate, fenofibrate, gemfibrozil, cerivastatin, pravastatin, fluvastatin, lovastatin, atorvastatin, simvastatin, xantinol, metoclopramide, amitriptyline, dibenzepine, venlafaxine, thioridazine, oxazepam, lithium, nitrofurantoin, dry plant extract, ascorbic acid and potassium, and/or the salts thereof used pharmaceutically.

Important active substances for transdermal therapeutic systems are, in particular, nicotine, glyceryl trinitrate, scopolamine, clonidine, fentanyl, estradiol, testosterone, oxibutynin, diclophenac, dexoyribonucleic acids for vaccines, for example, ibuprofen, ketoprofen, diltiazem, propranolol, albuterol, alprazolam, amethocaine, atenolol, benzoporphyrin, buprenorphine, calcitonin, dithranol, diphencypron, skin-penetrating peptides or peptides absorbable through the skin, eptazocine, ethinylestradiol, methrotrexate or naloxone.

Customary proportions for active pharmaceutical substances are from 10 to 100% by weight based on component (a).

Plasticizers

The addition of plasticizer allows physical properties to be adapted to the requirements of the individual drug forms, so that adhesive forces which are sufficient at room temperature and/or body temperature are obtained. Plasticizers in the stated proportions are able to lower the melt viscosity of the polymers employed in the liquid state. At room temperature, softening effects are evident. Influences on the release characteristics of embedded active substances are possible. A plasticizer should be present at not more than 30% by weight, preferably not more than 20, based on component (a). With particular preference no plasticizer is present.

Substances suitable as plasticizers generally have a molecular weight of between 100 and 20 000 and contain one or more hydrophilic groups in the molecule, e.g., hydroxyl, ester or amino groups. Suitability is possessed by citrates, phthalates, sebacates, and castor oil. Examples of suitable plasticizers are citric acid alkyl esters, glycerol esters, phthalic acid alkyl esters, sebacic acid alkyl esters, sucrose esters, sorbitan esters, dibutyl sebacate, and polyethylene glycols 4000 to 20 000. Preferred plasticizers are tributyl citrate, triethyl citrate, acetyl triethyl citrate, dibutyl sebacate, and diethyl sebacate. The amounts used are between 1 and 35%, preferably from 2 to 10% by weight, based on the (meth)acrylate copolymer.

Customary Pharmaceutical Additives

Customary pharmaceutical additives may be the following: mention may be made here, for example, of stabilizers, dyes, antioxidants, wetting agents, pigments, luster agents, etc. They serve primarily as processing auxiliaries and are intended to can ensure a reliable and reproducible production process and also good long-term storage stability. Further customary pharmaceutical excipients can be present in amounts of from 0.001% by weight to 100% by weight, preferably from 0.1 to 50% by weight, based on the copolymer. Examples of dry standardizers are: aluminum oxide, magnesium oxide, kaolin, talc, silica (Aerosils), barium sulfate, carbon black, and cellulose. Examples of release agents (mold release agents) are: esters of fatty acids or fatty acid amides, long-chain aliphatic carboxylic acids, fatty alcohols and their esters, montan waxes or paraffin waxes, and metal soaps; particular mention should be made of glyceryl monostearate, stearyl alcohol, glycerol behenic esters, cetyl alcohol, palmitic acid, carnauba wax, beeswax, etc.

Variations to the composition allow any unwanted effects of additions to the drug form to be compensated. The adhesives and binders of the invention may optionally include further additives in small amounts, if required by the specific formulation: neutral polymers, tackifiers, stabilizers, dyes, antioxidants, wetting agents, pore formers, humectants, complexing agents, etc.

Production Process:

The production of the transdermal therapeutic system depends on the form in which the polymer is employed: solid substances can be used directly by mixing with the additives in suitable mixers, compounders or extruders, which are heatable and possibly evacuable. The extruder is of single-screw or, preferably, twin-screw type in order that suitable mixing and transport properties are achieved.

The processing temperature is guided by the melting properties of the materials and is preferably between 20° C. and 200° C. Limiting factors are the thermal stability of the ingredients. Solid additives can be mixed with the polymer prior to extrusion. Liquid additives are added approximately halfway along the extrusion of the melt and produce a reduction in viscosity and a lowering of the temperature.

Polymer solutions or dispersions are admixed with the additives so that the latter are dissolved or suspended. The binder is obtained from these solutions, dispersions or suspensions by drying to form thin film layers.

Processing:

Coating, granulation, encapsulation or embedding take place by means of organic solution or aqueous dispersion of suitable auxiliaries. The use of melts is limited to substances having defined melting points in the range of the processing temperatures. Normally, low melt viscosities are required for processing.

In one version of the process the solid adhesive and binder of the invention is mixed with the powders and the mixture is mixed with a suitable solvent or the components are jointly melted.

Preferably from solution or suspension, or directly from the melt, coating out of the system onto sheetlike supports, e.g., films, fabrics or nonwovens, and drying or cooling give adhesive layers which fix the system on the skin and which owing to their hydrophilicity are particularly compatible. Coating takes place discontinuously in the laboratory, by means of a doctor blade, and continuously on the pilot plant and production scales, by means of rolling doctors or by roll application. Immediately after coating, a topsheet is added which is of low adhesion, is often siliconized, and is removed prior to use.

The assemblies or adhesive layers obtained can be processed further to drug forms for use. In this case it is possible to incorporate drugs as early as during the preparation of the adhesive and binder. In that case these active substances are fixed in particulate or dissolved form. Influencing the release of active substance by means of the adhesive and binder is a possibility and can be exploited for the formulation of drug forms.

Drug Forms

The drugs used for the purposes of the invention are intended to be used on or in the human or animal body in order

1. to heal, alleviate, prevent or diagnose diseases, ailments, physical damage or pathological symptoms.

2. to allow the state, condition or functions of the body, or mental states, to be identified.

3. to replace active substances or body fluids produced by the human or animal body.

4. to defend against, eliminate or render innocuous pathogens, parasites or exogenous substances, or

5. to influence the state, condition or functions of the body, or mental states.

Drugs in common use can be found in reference works, such as the Rote Liste or the Merck Index, for example.

In accordance with the invention it is possible to use any active substances which fulfill the desired therapeutic action in the sense of the above definition.

Drug forms can be produced by standard processing techniques from the intermediates prepared in accordance with the invention.

Supports coated with the adhesive and binder are generally in the form of rolls, protected by means of topsheets (release liners). From these continuous sheets it is possible for individual patches of the required size to be cut or punched and packaged individually.

The coating of sheetlike supports with polymer-containing liquids is described for example in Mass, J. and Schmidt, H.: Coating Technology for Transdermal Drug Delivery Systems, Medical Device Technology, 3/41990 edition, pp. 46-50.

Properties relevant for administration, required tests, and specifications are listed in pharmacopeias.

Details can be found in the standard text books, e.g.:

- Voigt, R. (1984): Lehrbuch der pharmazeutischen Technologie; Verlag Chemie Weinheim—Beerfield Beach/Florida—Basle.

- Sucker, H., Fuchs, P., Speiser, P.: Pharmazeutische Technologie, Georg Thieme Verlag Stuttgart (1991), especially chapters 15 and 16, pp. 626-642.

- Gennaro, A.,R. (editor), Remington's Pharmaceutical Sciences, Mack Publishing Co., Easton Pennsylvania (1985), Chapter 88, pp. 1567-1573.

- Heilmann, K.: Therapeutische Systeme, Ferdinand Euler Verlag, Stuttgart, pp. 52-57.

- Brandau, R. and Lippold, B.H. (1982): Dermal and Transdermal Absorption. Wissenschaftliche Verlags-gesellschaft mbH, Stuttgart, pp. 171-200.

### EXAMPLES

Implementation of the examples:

EUDRAGIT® E100: copolymer of 25% by weight methyl methacrylate, 25% by weight butyl methacrylate, and 50% by weight dimethylaminoethyl methacrylate.

PLASTOID® B: copolymer of 50% by weight methyl methacrylate, and 50% by weight butyl methacrylate. Durotak® 280 2516: polyacrylate adhesive (ingredients according to Rote Liste: acrylate/vinyl acetate/meth-acrylate copolymer)

### EXAMPLE 1

100 g of EUDRAGIT® E100 (copolymer of 25% by weight methyl methacrylate, 25% by weight butyl methacrylate, and 50% by weight dimethylaminoethyl methacrylate) are dissolved with stirring in a mixture of 53.6 g of acetone, 29.8 g of ethanol, and 6.0 g of isopropanol. 40 g of dibutyl sebacate, 12 g of succinic acid, and 16 g of alprazolam are incorporated into this solution. A layer of the adhesive 160 μm thick is dried at 60° C. for 10 minutes on an aluminum foil 50 μm thick. The resulting layer is clear and about 100 μm thick.

### EXAMPLE 2

100 g of EUDRAGIT® E100 (copolymer of 25% by weight methyl methacrylate, 25% by weight butyl methacrylate, and 50% by weight dimethylaminoethyl methacrylate) are dissolved with stirring in a mixture of 53.6 g of acetone, 29.8 g of ethanol, and 6.0 g of isopropanol. 40 g of dodecanol, 12 g of succinic acid, and 16 g of alprazolam are incorporated into this solution. A layer of the adhesive 160 μm thick is dried at 60° C. for 10 minutes on an aluminum foil 50 μm thick. The resulting layer is clear and about 100 μm thick.

### EXAMPLE 3

100 g of EUDRAGIT® E100 (copolymer of 25% by weight methyl methacrylate, 25% by weight butyl methacrylate, and 50% by weight dimethylaminoethyl methacrylate) are dissolved with stirring in a mixture of 53.6 g of acetone, 29.8 g of ethanol, and 6.0 g of isopropanol. 40 g of decanol, 9 g of succinic acid, and 16 g of alprazolam are incorporated into this solution. A layer of the adhesive 160 μm thick is dried at 60° C. for 10 minutes on an aluminum foil 50 μm thick. The resulting layer is clear and about 100 μm thick.

### EXAMPLE 4

100 g of EUDRAGIT® E100 (copolymer of 25% by weight methyl methacrylate, 25% by weight butyl methacrylate, and 50% by weight dimethylaminoethyl methacrylate) are dissolved with stirring in a mixture of 53.6 g of acetone, 29.8 g of ethanol, and 6.0 g of isopropanol. 20 g of decanol, 20 g of dibutyl sebacate, 12 g of succinic acid, and 16 g of alprazolam are incorporated into this solution. A layer of the adhesive 160 μm thick is dried at 60° C. for 10 minutes on an aluminum foil 50 μm thick. The resulting layer is clear and about 100 μm thick.

### EXAMPLE 5

100 g of EUDRAGIT® E100 (copolymer of 25% by weight methyl methacrylate, 25% by weight butyl methacrylate, and 50% by weight dimethylaminoethyl methacrylate) are dissolved with stirring in a mixture of 53.6 g of acetone, 29.8 g of ethanol, and 6.0 g of isopropanol. 50 g of myristyl alcohol, 9 g of succinic acid, and 16 g of alprazolam are incorporated into this solution. A layer of the adhesive 160 μm thick is dried at 60° C. for 10 minutes on an aluminum foil 50 μm thick. The resulting layer is clear and about 100 μm thick.

### EXAMPLE 6

100 g of PLASTOID® B are dissolved with stirring in a mixture of 53.6 g of acetone, 29.8 g of ethanol, and 6.0 g of isopropanol. 40 g of dodecanol, alprazolam are incorporated into this solution. A layer of the adhesive 160 μm thick is dried at 60° C. for 10 minutes on an aluminum foil 50 μm thick. The resulting layer is clear and about 100 μm thick. Example 7

100 g of PLASTOID® B are dissolved with stirring in a mixture of 53.6 g of acetone, 29.8 g of ethanol, and 6.0 g of isopropanol. 50 g of decanol, alprazolam are incorporated into this solution. A layer of the adhesive 160 μm thick is dried at 60° C. for 10 minutes on an aluminum foil 50 μm thick. The resulting layer is clear and about 100 μm thick.

### EXAMPLE 8

100 g of Durotak® 280 2515 are mixed with 30 g of decanol.

A layer of the adhesive 160 μm thick is dried at 60° C. for 10 minutes on an aluminum foil 50 μm thick. The resulting layer is clear and about 100 μm thick.

Measurement Methods

Determination of the Tack:

A group of testers applies a patch measuring about 16 cm² to the skin and makes an assessment in accordance with the following classification: 0 =the system does not adhere to the skin, even after 1 minute of gentle pressing. 1 =the system adheres to the skin with not more than 1 minute of gentle pressing 2 =the system adheres to the skin without additional pressing.

Determination of the Cold Flow:

A group of testers applies a patch measuring about 16 cm² to the skin and wears it under constant conditions over 24 hours. Thereafter the movement or slippage of the patch, in mm is measured.

Determination of the Fracture Behavior (Adhesive or Cohesive Fracture) and the Stickiness:

The determination is made by peeling a coated strip 50 mm wide and preferably of aluminum from a stainless steel plate and at the same time measuring the force required to achieve peel removal.

Determination of the penetration:

The determination was made using a modified Franz cell and human skin.

TABLE
Results of examples 1 to 8:
(Examples 2 to 4 = inventive; examples 1, 5 to 7, 8 = comparative
examples)

| | Example No. | | | | | | | |
|---|---|---|---|---|---|---|---|---|
| | 1 | 2 | 3 | 4 | 5 | 6 | 7 | 8 |
| Eudragit E100 | 100 | 100 | 100 | 100 | 100 | | | |
| Plastoid B | | | | | | 100 | 100 | |
| Durotak 280 2516 | | | | | — | | | 100 |
| Succinate | 12 | 12 | 9 | 12 | 9 | | | |
| Dibutyl sebacate | 40 | — | — | 20 | — | — | — | — |
| Decanol | | | 40 | 20 | | | 50 | 50 |
| Dodecanol | — | 40 | | | | 40 | | |
| Myristyl alcohol | | | | | 50 | | | |
| Tack (target = min. 1) | 1 | 2 | 2 | 2 | 0 | 0 | 0 | 2 |
| Cold flow [mm/24 h] | 0 | 0.08 | 0.07 | 0.05 | — | — | — | 5 |
| (target − max. 1) | | | | | | | | |
| Adhesive fracture | + | + | + | + | + | + | + | − |
| (target = +) | | | | | | | | |
| Bond strength [cm/min] | 2.3 | 5.0 | 4.4 | 3.0 | 0 | 0 | 0 | n.d. |
| (target − min. 1) | | | | | | | | |
| Penetration [μg/cm²] | 0.5 | 1.9 | 2.8 | 2.4 | n.d. | n.d. | n.d. | n.d. |
| (target = min. 1) | | | | | | | | |

n.d. = not determined

1. An adhesive and binder for dermal or transdermal therapeutic systems, comprising
(a) a (meth)acrylate copolymer of free-radically polymerized C₁ to C₄ alkyl esters of acrylic or methacrylic acid and (meth)acrylate monomers having a cationic ammonium group in the alkyl radical, comprising
(b) 0.1-45% by weight based on (a) of an organic dicarboxylic or tricarboxylic acid or of an acid-functional acrylate or (meth)acrylate polymer or copolymer, and
(c) a skin penetration enhancer
(d) if desired, an active pharmaceutical substance, a plasticizer and/or one or more customary pharmaceutical additives wherein the penetration enhancer (c) is an alcohol having 10 to 12 carbon atoms.

2. The adhesive and binder of claim 1, characterized in wherein the penetration promoter (c) is decanol, undecanol or dodecanol.

3. The adhesive and binder of claim 1, wherein the cationic (meth)acrylate copolymer (a) is composed of from 30 to 80% by weight of free-radically polymerized C₁ to C₄ alkyl esters of acrylic or methacrylic acid and from 70 to 20% by weight of (meth)acrylate monomers having a tertiary ammonium group in the alkyl radical.

4. The adhesive and binder of claim 1, wherein the cationic (meth)acrylate copolymer (a) is composed of from 85 to 98% by weight of free-radically polymerized C₁ to C₄ alkyl esters of acrylic or methacrylic acid and from 15 to 2% by weight of (meth)acrylate monomers having a quaternary ammonium group in the alkyl radical.

5. The adhesive and binder of claim 1, wherein as organic dicarboxylic tricarboxylic acids (b) succinic acid (succinate), fumaric acid or citric acid are present.

6. The adhesive and binder of claim 1, wherein as acid-functional acrylate or (meth)acrylate polymer (b) a copolymer of 30-70% by weight of ethyl acrylate or methyl methacrylate and 70 30% by weight of methacrylic acid is present.

7. The adhesive and binder of claim 1, wherein as acid-functional acrylate or (meth)acrylate polymer (b) polyacrylic acid is present.

8. The adhesive and binder of claim 1, wherein the cationic radicals of the cationic (meth)acrylate copolymer (a) are neutralized to an extent of 2-100% by means of component (b).

9. The adhesive and binder of claim 1, wherein as active pharmaceutical substance an active substance from the therapeutic categories of the analgesics, antirheumatics, antiallergics, antiarrhythmics, beta-receptor blockers, calcium channel blockers, inhibitors of the renin-angiotensin system, broncholytics/antasthmatics, cholinergics, diuretics, circulation promoters, gout agents, influenza agents, coronary agents, lipid reducers, gastrointestinal agents, psychopharmaceuticals, platelet aggregation inhibitors, urological agents, venous therapeutic agents, vitamins or minerals is present.

10. The adhesive and binder of claim 9, wherein as active pharmaceutical substance morphine and/or its derivatives, tramadol, acetylsalicylic acid, diclofenac, indometacin, lonazolac, ibuprofen, ketoprofen, propyphenazone, naproxen, paracetamol, flurbiprofen, dimetindene, quinidine, metoprolol, propranolol, oxprenolol, pindolol, atenolol, metoprolol, disopyramide, verapamil, diltiazem, gallopamil, nifedipine, nicardipine, nisoldipine, nimodipine, amlodipine, theophylline, salbutamol, terbutaline, ambroxol, aminophylline, choline theophyllinate, pyridostigmine, piretanide, furosemide, pentoxifylline, naftidrofuryl, buflomedil, xantinol nicotinate, bencyclane, allopurinol, norephedrine, clorphenamine isosorbide mononitrate, isosorbide dinitrate, glyceryl trinitrate, molsidomine, bezafibrate, fenofibrate, gemfibrozil, cerivastatin, pravastatin, fluvastatin, lovastatin, atorvastatin, simvastatin, xantinol, metoclopramide, amitriptyline, dibenzepine, venlafaxine, thioridazine, oxazepam, lithium, nitrofurantoin, dry plant extract, ascorbic acid and potassium, and/or the salts thereof used pharmaceutically is present.

11. The adhesive and binder of claim 9, wherein as active pharmaceutical substance nicotine, glyceryl trinitrate, scopolamine, clonidine, fentanyl, estradiol, testosterone, oxibutynin, diclophenac, dexoyribonucleic acids for vaccines, for example, ibuprofen, ketoprofen, diltiazem, propranolol, albuterol, alprazolam, amethocaine, atenolol, benzoporphyrin, buprenorphine, calcitonin, dithranol, diphencypron, skin-penetrating peptides or peptides absorbable through the skin, eptazocine, ethinylestradiol, methrotrexate or naloxone is present.

12. A process for producing an adhesive and binder of by combining components (a) to (c) and, if desired, (d) with one another, with or without the addition of water, and converting them into the coating agent and binder by means of melting, injection molding, extrusion, casting, coating out, spraying or compressing.
